(19) United States
(12) Patent Application Publication
(10) Pub. No.: US 2010/0228237 A1
(43) Pub. Date: Sep. 9, 2010

(54) GOLD NANOCAGES CONTAINING MAGNETIC NANOPARTICLES

(75) Inventors: Bong Hyun Chung, Daejeon (KR); Yong Taik Lim, Daejeon (KR); Jin Kyeong Kim, Daejeon (KR)

Correspondence Address: MOORE & VAN ALLEN PLLC, P.O. BOX 13706, Research Triangle Park, NC, 27709, US

(73) Assignee: Korea Research Institute of Bioscience and Biotecnology, Daejeon (KR)

(21) Appl. No.: 12/376,792

(22) PCT Filed: Jul. 30, 2007
(86) PCT No.: PCT/KR2007/003658

§ 371 Date: Jun. 18, 2009

(30) Foreign Application Priority Data
Aug. 8, 2006 (KR) 10-2006-0074748

(51) Int. Cl. A61B 18/20; B05D 5/12; B05D 7/00; B82B 3/00; C22C 5/02; B32B 1/00

(52) U.S. Cl. 606/13; 427/127; 427/216; 420/507; 428/402; 428/364; 428/403; 977/810; 977/890; 977/915

(57) ABSTRACT

The present invention relates to gold nanocages containing magnetic nanoparticles and a preparation method thereof. More specifically, relates to hollow-type gold nanocage particles, which contain iron oxide nanoparticles having a magnetic property and have an optical property of strongly absorbing or scattering light in the near-infrared (NIR) region, as well as a preparation method thereof. Due to their optical property and magnetic property, the magnetic nanoparticle-containing gold nanocages can be used in various applications, including analysis in a turbid medium with light, cancer therapy or biomolecular manipulation using light, contrast agents for magnetic resonance imaging, magnetic hyperthermia treatment and drug delivery guide, etc.

### TECHNICAL FIELD

The present invention relates to gold nanocages containing magnetic nanoparticles and a preparation method thereof, and more particularly to hollow-type gold nanocage particles, which contain iron oxide nanoparticles having a magnetic property and have an optical property of strongly absorbing or scattering light in the near-infrared (NIR) region, as well as a preparation method thereof.

### BACKGROUND ART

Gold or silver nanoparticles exhibit strong surface plasmon resonances (light absorption or scattering) at specific wavelengths based on the sizes and shapes thereof. Also, these nanoparticles have very excellent optical stability compared to that of common organic dyes, and the surface plasmon resonance frequency thereof can also be controlled by changing the size, shape or structure thereof (Jin, R. et al., Science, 294:1901, 2000).

Using such properties of metal nanoparticles, studies on metal nanoparticles are being actively conducted in various fields, including biosensors for sensing biomaterials such as DNA or proteins, metal nanoparticle-containing detectors for detecting biomaterials (WO 2005/047864), surgical prosthetic biomaterials containing metal nanoparticles (U.S. 60/458,227), and chemical sensors comprising encapsulated metal nanoparticles (KR 10-2005-0065904).

Recently, biosensors containing gold nanoparticles have been reported. For example, WO 2006/021091 discloses a biosensor for targeting specific DNA depending on the size of gold nanoparticles. Meanwhile, although metal nanoparticles may be used as test nanoparticle colloidal solutions, they can be used as tools for surface enhanced Raman scattering (SERS) after being coated onto a specific substrate or can be used as various biological and chemical sensors by forming an array thereof or coating them on colloidal particles (Taton, T. A. et al., Science, 289:1757, 2000).

However, light in the visible region cannot be used to immediately analyze turbid samples such as blood or the skin without a separate purification process or to apply them to the human body. Thus, in order to widen the application range of the metal nanoparticles, it is required to prepare metal nanoparticles having the property of strongly absorbing or scattering light in the near-infrared region in which light transparency is the highest (Weissleder, R. et al., Nature Biotechnology, 17:375, 1999).

For this reason, the preparation of nanoparticles such as metal nanorods, metal nano-core-shells, metal nanocubes and metal nanocages, which exhibit optical properties in the near-infrared region, has recently been performed (US 20010002275A1, Averitt, R. D. et al., Physical Review Letters, 78:4217, 1997).

However, the fundamental limitations of optical analysis methods also exist in the analysis methods or applications that use near-infrared properties. Specifically, because the penetration depth of light cannot exceed a maximum depth of 1 cm, there have been many limitations in the analysis methods or applications that use near-infrared properties. Currently, as solutions capable of overcoming such limitations of optical methods, a method that uses magnetic properties, and a radio-nuclear, medical method, are being suggested. However, such two methods do not have the advantages of the optical methods.

Thus, in order to solve the above-described problems, there is an urgent need to develop technologies for multifunctional nanoparticles, which exhibit not only an optical property of strongly absorbing near-infrared light, but also a magnetic property.

Accordingly, the present inventors have made many efforts to solve the above-described problems occurring in the prior art that uses metal nanoparticles. As a result, the present inventors have prepared gold nanonanocage particles containing magnetic nanoparticles by sequentially coating a gold nanolayer and a silver nanolayer on magnetic nanoparticles, and then forming gold shells thereon, and found that the gold nanocages exhibit not only an optical property of absorbing or scattering light in the near-infrared region, but also a magnetic property, thereby completing the present invention.

### SUMMARY OF THE INVENTION

It is a main object of the present invention to provide gold nanocages containing magnetic nanoparticles and a preparation method thereof.

Another object of the present invention is to provide gold nanocages containing biomaterials and a preparation method thereof.

To achieve the above objects, in one aspect, the present invention provides gold nanocages, which contain magnetic nanoparticles and has an optical property of absorbing or scattering light in the near-infrared region.

In another aspect, the present invention provides a method for preparing gold nanocages containing magnetic nanoparticles, the method comprising the steps of: (a) coating magnetic nanoparticles with gold; (b) coating the gold-coated magnetic nanoparticles with silver; and (c) incorporating gold ions (HAuCl₄) into the silver-coated magnetic nanoparticles under reflux.

In still another aspect, the present invention provides biomaterial-containing gold nanocages, in which a biomaterial selected from the group consisting of antibodies, ligands, peptides and proteins is coated on or bound to gold nanocages, as well as a preparation method thereof.

Other features and aspects of the present invention will be apparent from the following detailed description and the appended claims.

### BRIEF DESCRIPTION OF DRAWINGS

FIG. 1 schematically shows a process for preparing gold nanocages containing iron oxide nanoparticles.

FIG. 2 illustrates electron microscope photographs of iron oxide nanoparticles (1), gold-coated iron oxide nanoparticles (2), iron oxide nanoparticles (3) comprising silver coated on the nanoparticles (2), and iron oxide nanoparticle-containing gold nanocages (4), prepared through the process shown in FIG. 1.

FIG. 3 shows the absorption spectra of the nanoparticles shown in FIG. 2.

FIG. 4 shows the absorption spectra of gold nanocages having various sizes.

FIG. 5 shows an MRI image of gold-coated iron oxide nanoparticles.

FIG. 6 shows an MRI image of gold nanocage particles containing iron oxide nanoparticles.

FIG. 7 is a photograph showing that breast cancer cells (SKBR-3) were killed with a near-infrared laser (810 nm) using antibody-coated, iron oxide nanoparticle-containing gold nanocages.

### DETAILED DESCRIPTION OF THE INVENTION, AND PREFERRED EMBODIMENTS

In one aspect, the present invention relates to gold nanocages, which contain magnetic nanoparticles and have an optical property of absorbing or scattering light in the near-infrared region.

In the present invention, the gold nanocages are preferably hollow-type gold nanostructures. The gold nanocages preferably comprise gold shells. The gold shells preferably have a thickness of 1-1000 nm.

In the present invention, the shape of the gold nanocages is preferably selected from the group consisting of spheres, rods, cubes, prisms, pyramids and triangles, but the scope of the present invention is not limited thereto.

In the present invention, the magnetic nanoparticles are preferably Fe₂O₃ or Fe₃O₄, and the shape of the magnetic nanoparticles is preferably selected from the group consisting of nanospheres, nanorods and nanocubes.

In the present invention, the magnetic nanoparticles preferably have a metal coated thereon, in which the metal is preferably gold or silver.

In another aspect, the present invention relates to a method for preparing gold nanocages containing magnetic nanoparticles, the method comprising the steps of: (a) coating magnetic nanoparticles with gold; (b) coating the gold-coated magnetic nanoparticles with silver; (c) incorporating gold ions (HAuCl₄) into the silver-coated magnetic nanoparticles under reflux.

In the present invention, the magnetic nanoparticles are preferably iron oxide nanoparticles.

In still another aspect, the present invention relates to biomaterial-containing gold nanocages, in which a biomaterial selected from the group consisting of antibodies, ligands, peptides and proteins is coated on or bound to the gold nanocages.

In yet another aspect, the present invention relates to a method for preparing biomaterial-containing gold nanocages, the method comprises coating or binding a biomaterial selected from the group consisting of antibodies, ligands, peptides and proteins to said gold nanocages.

In the present invention, the gold nanocages, which contain magnetic nanoparticles and have an optical property of absorbing or scattering light in the near-infrared region, are multifunctional metal nanoparticles, which have not only an optical property of absorbing near-infrared light, but also the magnetic property of the magnetic nanoparticles.

In the present invention, the gold nanocages that strongly absorb near-infrared light serve to selectively destroy tissues such as skin cancer around the skin using near-infrared light that can penetrate deep into the skin. The gold nanocages have an advantage in that they can greatly reduce side effects or pain, because the gold nanocages absorb near-infrared light, convert the absorbed light into thermal energy, and then selectively kill only target cancer cells (see FIG. 7).

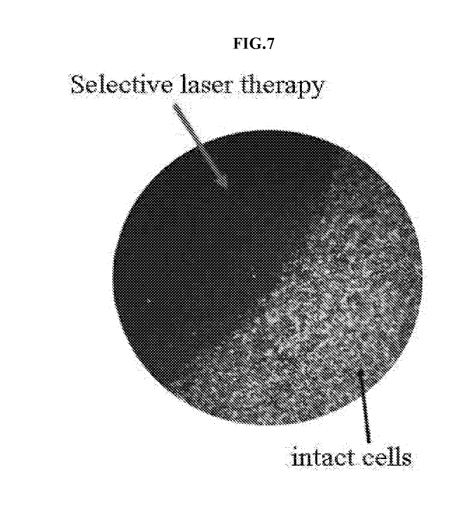

The optical property of the magnetic nanoparticle-containing gold nanocages of the present invention enables the gold nanocages to be used as contrast agents for magnetic resonance imaging (MRI) as shown in FIG. 6.

Also, the magnetic property of the magnetic nanoparticle-containing gold nanocages of the present invention can guide the gold nanocage particles to the desired site in the human body and enables the gold nanocages to be used in various applications such as magnetic hyperthermia treatment.

Also, the magnetic property and optical property of the magnetic nanoparticle-containing gold nanocages according to the present invention can be applied such that the gold nanocages target specific cells or tissue, and then selectively kill only specific cells and biomolecules.

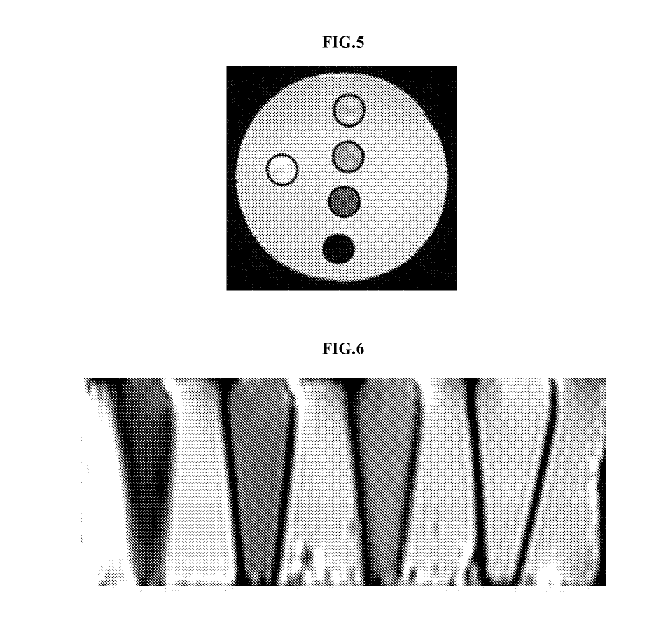

The gold nanocages of the present invention absorb or scatter light in the near-infrared region, in which the absorption and scattering spectra are preferably in the range of 600-2000 nm, and the wavelength range of the spectra vary depending on the average particle size of the gold nanocages (see FIG. 4). The particle size of the gold nanocages is not limited to a few nanometers (nm), but rather can be enlarged to the range from a few tens of nanometers (nm) to a few hundreds of nanometers (nm).

In one embodiment of the present invention, in order to realize the magnetic property of the magnetic nanoparticle-gold nanocages, spherical iron oxide nanoparticles are selected as the magnetic nanoparticles. However, the scope of the present invention is not limited thereto, and it is possible to use various kinds of metal nanoparticles having magnetic properties and to use various shapes of magnetic nanoparticles, including nanorods and nanocubes.

Also, due to the various shapes of the magnetic nanoparticles selected as a base material, the resulting gold nanocages also have various shapes, including sphere, rod, cube, prism, pyramid and triangle.

In order to shift the light absorption spectrum of the magnetic nanoparticle-containing gold nanocages to the long-wavelength region, the present invention provides hollow-type gold nanocages. To facilitate the formation of the hollow-type gold nanocages, metal nanolayers are coated on the magnetic nanoparticles.

In one embodiment of the present invention, the metal nanolayers are formed by sequentially coating a gold nanolayer and a silver nanolayer on the magnetic nanoparticles. However, the scope of the present invention is not limited thereto, various metal nanolayers can be coated on the magnetic nanoparticles, and the shape of the metal nanolayers can vary depending on the shape of the magnetic nanoparticles.

Then, to form gold shells, HAuCl₄ is added to the magnetic nanoparticles having the gold nanolayer and the silver nanolayer sequentially coated thereon under reflux. To the solution, NaCl is added to remove AgCl produced after the reaction of HAuCl₄ with the silver nanolayer so as to form hollow regions and gold shells, thus preparing gold nanocages containing magnetic nanoparticles.

In addition, the magnetic nanoparticle-containing gold nanocages prepared as described above can be coated with a biomaterial, such that they can be applied in various biological and medical fields. In one embodiment of the present invention, the biomaterial is cancer-specific biomaterial selected from the group consisting of cancer-specific antibodies, ligands, peptides and proteins; however, the scope of the present invention is not limited thereto, biomaterials associated with various diseases can be used in the present invention.

### EXAMPLES

Hereinafter, the present invention will be described in further detail with reference to examples. It will be obvious to those skilled in the art that these examples are illustrative only, and the scope of the present invention is not limited thereto.

Particularly, although only iron oxide nanoparticles were illustrated as magnetic nanoparticles in the following examples, it will be obvious to those skilled in the art that various metal nanoparticles having magnetic properties may also be used.

Also, although only spherical magnetic nanoparticles were illustrated in the following examples, it will be obvious to those skilled in the art that magnetic nanoparticles having various shapes, such as nanospheres, nanorods and nanocubes, may also be used. In addition, it will also be obvious to those skilled in the art that the shape of the resulting gold nanocages can be controlled depending on the shape of the magnetic nanoparticles.

Furthermore, although the following examples illustrated an antibody as a biomaterial coated on gold nanocages containing magnetic nanoparticles, it will be obvious to those skilled in the art that it is possible to use not only cancer-specific biomaterials, such as cancer-specific antibodies, ligands, peptides and proteins, but also biomaterials associated with various diseases.

### Example 1

### Preparation of Gold-Coated Iron Oxide Nanoparticles (Fe₃O₄@Au)

To 20 ml of benzyl ether, 0.71 g (2 mmol) of iron(III) acetylacetonate, 2 ml (6 mmol) of oleic acid, 2 ml (0-4 mmol) of oleylamine and 2.58 g (10 mmol) of 1,2-hexadecanediol were added, and the mixture solution was rapidly stirred in an argon atmosphere.

After the solution was allowed to react at 200° C. for 2 hours, argon gas was removed, followed by reaction at 290° C. for 1 hour. After completion of the reaction, the solution was cooled at room temperature and washed several times with ethanol. Finally, Fe₃O₄ was separated from the solution using a magnet, and 0.1 g of the separated Fe₃O₄ was dispersed in 40 ml of benzyl ether.

To the dispersion, 0.7 g (2.2 mmol) of gold(III) acetate, 3.1 g (12 mmol) of 1,2-hexadecanediol, 0.5 ml (0-1.5 mmol) of oleic acid and 3 ml (0-6 mmol) of oleylamine were added, and the mixture solution was rapidly stirred in an argon atmosphere, while it was allowed to react at 190° C. for 1.5 hours. After completion of the reaction, the solution was cooled at room temperature and washed several times with ethanol.

Finally, Fe₃O₄ was separated from the solution using a magnet, thus preparing gold-coated iron oxide nanoparticles (Fe₃O₄@Au). An MRI photograph of the prepared gold-coated iron oxide nanoparticles is shown in FIG. 5.

### Example 2

### Preparation of Silver-Coated Iron Oxide Nanoparticles (Fe₃O₄@Au@Ag)

Fe₃O₄@Au prepared in Example 1 was dispersed in 100 ml of hexane, and the dispersion was mixed with 10 mM MUA (mercaptoundecanoic acid). The mixture solution was sonicated for 1 hour, and then Fe₃O₄ was separated from the solution using a magnet. The separated Fe₃O₄ was washed several times with ethanol, and then dispersed in 100 ml of triple-deionized water.

The dispersed solution was adjusted to a pH of 10 by the addition of 100 mM NaOH, and then diluted 10-fold by adding 9 ml of triple-deionized water to 1 ml of the solution. After the solution was adjusted to a pH of 10 by the addition of 100 mM NaOH, and then 0.5 ml of 100 mM AgNO₃ was added thereto. The solution was refluxed with rapid stirring at 100° C., and then 1 ml of 50 mM sodium citrate was added thereto dropwise within 1 minute. The solution was allowed to react further for 20 minutes, thus preparing iron oxide nanoparticles (Fe₃O₄@Au@Ag) comprising silver coated on the nanoparticles prepared in Example 1.

### Example 3

### Preparation of Iron Oxide Nanoparticle-Containing Gold Nanocages (Fe₃O₄@Au@Au)

The Fe₃O₄@Au@Ag solution prepared in Example 2 was centrifuged three times at 10000 rpm for 10 minutes each time, and then unreacted sodium citrate was washed out. The solution was dispersed in 10 ml of triple-distilled water, and 100 mg of PVP (polyvinylpyrolidone) was dissolved therein. The mixture solution was refluxed with rapid stirring at 100° C., and then 0.8 ml of 10 mM HAuCl₄ was added thereto dropwise at a constant rate of 0.425 ml/min.

After the addition of HAuCl₄, the solution was allowed to react for 20 minutes to stabilize. Then, the reaction solution was cooled at room temperature, and an excess amount of NaCl was added thereto to remove white AgCl, thus producing hollow-type nanostructures. The resulting solution was centrifuged three times at 10000 rpm for 10 minutes each time, and then gold shells were collected from the solution using a magnet, thus preparing iron oxide nanoparticle-containing gold nanocages (Fe₃O₄@Au@Au). An MRI photograph of the prepared iron oxide nanoparticle-containing gold nanocages is shown in FIG. 6.

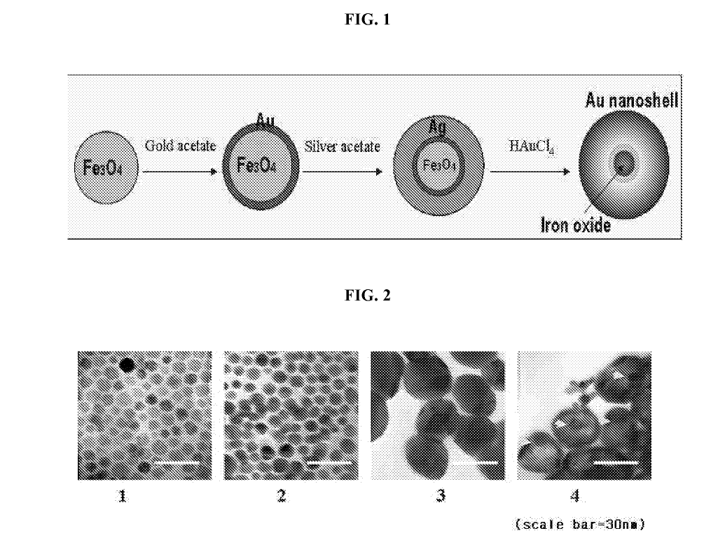

The process of Examples 1-3 for preparing iron oxide nanoparticle-containing gold nanocages is shown in FIG. 1. Also, FIG. 2 illustrates electron microscope photographs of iron oxide nanoparticles, and the gold-coated iron oxide nanoparticles, the iron oxide nanoparticles comprising silver coated on the nanoparticles, and the iron oxide nanoparticle-containing gold nanocages, which were prepared in Examples 1 to 3, respectively.

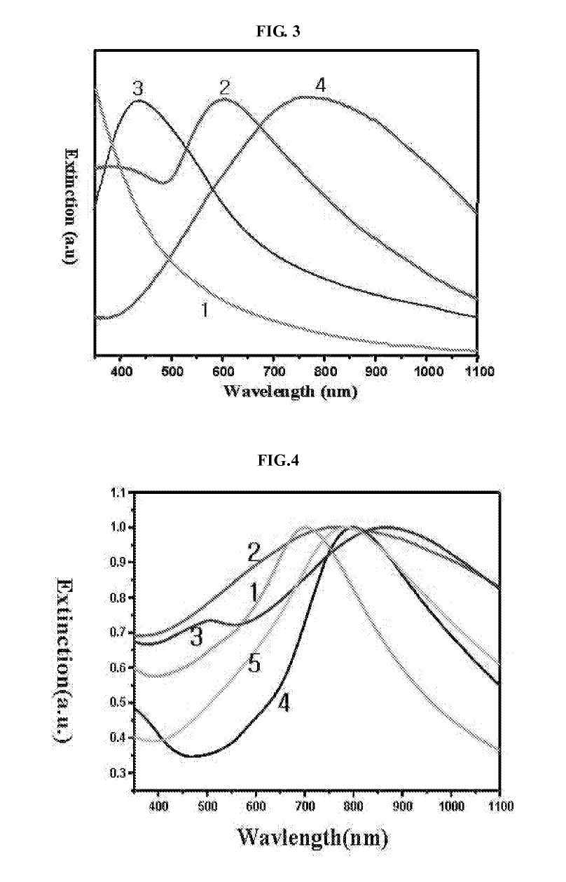

FIG. 3 shows the absorption spectra of the nanoparticles shown in FIG. 2. As can be seen in FIG. 3, iron oxide nanoparticles (1) did not show a specific absorption peak in the visible region, but with the beginning of gold coating (2), they showed a strong absorption peak at a wavelength of 600 nm. When the gold-coated nanoparticles were coated with a silver nanolayer (3), the nanoparticles showed an absorption peak at a wavelength of 430 nm, and when the silver nanolayer was changed to a hollow gold layer (4), the nanoparticles showed a strong absorption peak at a wavelength of 808 nm. From such optical properties, the structural change of the nanoparticles in each step of FIG. 2 could be confirmed.

FIG. 4 shows hollow-type gold nanostructures having various absorption peaks. The average particle size and shell thickness of the hollow-type gold nanostructures are shown in Table 1 below.

TABLE 1

| | 1 | 2 | 3 | 4 | 5 |
|---|---|---|---|---|---|
| Average | ca. 45 nm | ca. 52 nm | ca. 65 nm | ca. 76 nm | ca. 84 nm |
| particle size | | | | | |
| Shell | ca. 5 nm | ca. 5 nm | ca. 5 nm | ca. 5 nm | ca. 5 nm |
| thickness | | | | | |

### Example 4

### Preparation of Antibody-Coated Gold Nanocages

Fe₃O₄@Au@Au prepared in Example 3 was dispersed in 1 ml of medium provided an optical density (O.D.) of 2.8. 100 μL of cys-protein G (300 mg/ml) was added to the dispersion, and the mixture solution was slowly stirred at 4° C. for 12 hours.

To the stirred solution, 80 μL of NEU antibodies (200 mg/ml) was added, and the mixture solution was slowly stirred at 4° C. for 12 hours. Then, 5 μL of TRITC (tetramethylrhodamine isothiocyanate)-secondary antibodies (3000 mg/ml) was added thereto, and the mixture solution was slowly stirred at 4° C. for 12 hours.

Finally, 10 mg/ml of SH-PEG (thiol-polyethyleneglycol) was dissolved in the solution, and then the mixture was slowly stirred at 4° C. for 12 hours, thus preparing antibody-coated, magnetic nanoparticle-containing gold nanocages.

Using the antibody-coated, iron oxide nanoparticle-containing gold nanocages, prepared in Example 4, cells were irradiated by a near-infrared laser (810 nm). As a result, it could be seen that only breast cancer cells (SKBR-3) were selectively killed (see FIG. 7). In FIG. 7, the green portion is a portion stained with a dye staining only living cells. As can be seen in FIG. 7, among cells to which the gold nanocages were attached, only cells irradiated with the laser were killed.

### INDUSTRIAL APPLICABILITY

As described in detail above, the present invention provides magnetic nanoparticle-containing gold nanocages, which overcome the problems occurring in the prior optical methods and have not only an optical property, but also a magnetic property, as well as a preparation method thereof. Due to their optical property and magnetic property, the magnetic nanoparticle-containing gold nanocages according to the present invention can be used in various applications, including analysis in a turbid medium with light, cancer therapy or biomolecular manipulation using light, contrast agents for magnetic resonance imaging, magnetic hyperthermia treatment and drug delivery guide, etc.

While the present invention has been described in detail with reference to specific features, it will be apparent to those skilled in the art that this description is only for a preferred embodiment and does not limit the scope of the present invention. Thus, the substantial scope of the present invention will be defined by the appended claims and equivalents thereof.

1. Gold nanocages comprising magnetic nanoparticles which have an optical property of absorbing or scattering light in the near-infrared region.

2. The gold nanocages according to claim 1, comprising hollow-type gold nano structures.

3. The gold nanocages according to claim 1, comprising gold shells.

4. The gold nanocages according to claim 3, wherein the thickness of the gold shells is 1-1000 nm.

5. The gold nanocages according to claim 1, wherein the shape of the gold nanocages is selected from the group consisting of spheres, rods, cubes, prisms, pyramids and triangles.

6. The gold nanocages according to claim 1, wherein the magnetic nanoparticles comprise Fe₂O₃ or Fe₃O₄.

7. The gold nanocages according to claim 6, wherein the shape of the magnetic nanoparticles is selected from the group consisting of nanospheres, nanorods and nanocubes.

8. The gold nanocages according to claim 6, wherein the magnetic nanoparticles have a metal coated thereon.

9. The gold nanocages according to claim 8, wherein the metal is gold or silver.

10. A method for preparing gold nanocages containing magnetic nanoparticles, the method comprising the steps of:
(a) coating magnetic nanoparticles with gold;
(b) coating the gold-coated magnetic nanoparticles with silver; and
(c) incorporating gold ions (HAuCl₄) into the silver-coated magnetic nanoparticles under reflux.

11. The method for preparing gold nanocages according to claim 10, wherein the magnetic nanoparticles comprise iron oxide nanoparticles.

12. Biomaterial-containing gold nanocages, in which a biomaterial selected from the group consisting of antibodies, ligands, peptides and proteins is coated on or bound to the gold nanocages of claim 1.

13. A method for preparing biomaterial-containing gold nanocages, the method comprises coating or binding a biomaterial selected from the group consisting of antibodies, ligands, peptides and proteins to the gold nanocages of claim 1.

14. A method of treating a cancer, said method comprising:
targeting cells with the gold nanocages of claim 1; and
irradiating said gold nanocage-attached cells with light.

15. The method of claim 14, wherein the light is a laser.

16. The method of claim 14, wherein the light is near IR radiation.

17. A method of treating a cancer, said method comprising:
targeting cells with the gold nanocages of claim 12; and
irradiating said biomaterial-containing gold nanocage-attached cells with light.
